(19) United States
(12) Patent Application Publication
(10) Pub. No.: US 2021/0291231 A1
(43) Pub. Date: Sep. 23, 2021

(54) ELECTROMECHANICAL TRANSDUCER ELEMENT, ULTRASONIC TRANSDUCER, ULTRASONIC PROBE, ULTRASONIC DIAGNOSTIC APPARATUS, AND METHOD FOR MANUFACTURING ELECTROMECHANICAL TRANSDUCER ELEMENT

(71) Applicant: Ricoh Company, Ltd., Tokyo (JP)

(72) Inventors: Yuichi ANDO, Hyogo (JP); Shuya ABE, Hyogo (JP); Osamu MACHIDA, Osaka (JP)

(73) Assignee: Ricoh Company, Ltd., Tokyo (JP)

(21) Appl. No.: 17/204,296

(22) Filed: Mar. 17, 2021

(30) Foreign Application Priority Data
Mar. 23, 2020 (JP) 2020-051413
Mar. 12, 2021 (JP) 2021-040482

(51) Int. Cl. B06B 1/06; H01L 41/317; A61B 8/00

(52) U.S. Cl. CPC B06B 1/0651; B06B 1/0622; B06B 1/0666; B06B 2201/76; A61B 8/4444; A61B 8/4488; A61B 8/46; H01L 41/317

(57) ABSTRACT

An electromechanical transducer element includes a base substrate, a first electrode on the base substrate, a piezoelectric body on the first electrode, and a second electrode on the piezoelectric body. The base substrate has a void area opposite to the piezoelectric body via the first electrode, and a width of the void area on a cross section cut along a layer direction of the electromechanical transducer element satisfies 0.65≤Pw/Cw≤0.95, where Cw represents the width of the void area, and Pw represents a width of the piezoelectric body on the cross section.

### CROSS-REFERENCE TO RELATED APPLICATIONS

This patent application is based on and claims priority pursuant to 35 U.S.C. § 119(a) to Japanese Patent Application Nos. 2020-051413, filed on Mar. 23, 2020, and 2021-040482, filed on Mar. 12, 2021 in the Japan Patent Office, the entire disclosure of which is hereby incorporated by reference herein.

### BACKGROUND

### Technical Field

The present disclosure relates to an electromechanical transducer element, an ultrasonic transducer, an ultrasonic probe, an ultrasonic diagnostic apparatus, and a method for manufacturing the electromechanical transducer element.

### Related Art

Transducers that vibrate thin films to transmit and receive ultrasonic wave are used as inspection apparatuses and measurement apparatuses for medical diagnosis, industrial equipment use, in-vehicle equipment, and marine use.

In particular, medical ultrasonic diagnostic apparatuses are widely used because of easiness of real-time observation of internal tissues.

Conventionally, an electromechanical transducer element used for such an ultrasonic transducer is manufactured by dicing from a ceramic lead zirconate titanate (PZT) called bulk. In recent years, there are electromechanical transducer elements manufactured using semiconductor technologies such as piezoelectric micro-machined ultrasonic transducer (PMUT) using a piezo element and capacitive micro-machined ultrasonic transducer (CMUT).

In particular, when PMUT is used, the resolution and the frequency can be increased by minute processing, the manufacturing is relatively easy due to a simple structure, and operation is possible at relatively low voltage. Therefore, PMUT is expected as a technology suitable for compact or thin devices and two-dimensional arrangement.

### SUMMARY

An embodiment of the present disclosure provides an electromechanical transducer element that includes a base substrate, a first electrode on the base substrate, a piezoelectric body on the first electrode, and a second electrode on the piezoelectric body. The base substrate has a void area opposite to the piezoelectric body via the first electrode. On a cross section cut along a layer direction of the electromechanical transducer element, the void area has a width Cw that satisfies 0.65≤Pw/Cw≤0.95, where Pw represents a width of the piezoelectric body on the cross section.

### BRIEF DESCRIPTION OF THE DRAWINGS

A more complete appreciation of the disclosure and many of the attendant advantages and features thereof can be readily obtained and understood from the following detailed description with reference to the accompanying drawings, wherein:

FIG. 1 is a diagram illustrating an example of a configuration of an ultrasonic diagnostic apparatus according to an embodiment of the present disclosure;

FIG. 2 is a block diagram illustrating a configuration of a controller of the ultrasonic diagnostic apparatus illustrated in FIG. 1;

FIG. 3 is a diagram illustrating another example of the configuration of the ultrasonic diagnostic apparatus;

FIG. 4 is a diagram illustrating an example of a configuration of an ultrasonic probe, including an ultrasonic transducer, of the ultrasonic diagnostic apparatus illustrated in FIG. 1;

FIG. 5 is a perspective view illustrating an example of a configuration of the ultrasonic transducer illustrated in FIG. 4;

FIG. 6 is a view illustrating an example of a configuration of the ultrasonic transducer illustrated in FIG. 4;

FIG. 7 is a cross-sectional view illustrating an example of a piezoelectric element of the ultrasonic transducer illustrated in FIG. 4;

FIGS. 8A to 8E are diagrams illustrating an example of manufacturing processes of the ultrasonic transducer illustrated in FIG. 4;

FIG. 9 is a cross-sectional view of the ultrasonic transducer illustrated in FIG. 7 when vibrating;

FIG. 10 is an enlarged view of an inflection point in the cross-sectional view illustrated in FIG. 9;

FIG. 11 is a graph illustrating the frequency dependence of a void area of electromechanical transducer illustrated in FIG. 7;

FIG. 12 is a graph illustrating an example of relation between the shape (thickness) of a piezoelectric body and output sound pressure;

FIG. 13 is a graph illustrating an example of relation between the size of the piezoelectric body and output sound pressure;

FIG. 14 is a graph illustrating an example of relation between the size of the piezoelectric body and reception sensitivity;

FIG. 15 is a graph illustrating an example of relation between the size of an upper electrode of the piezoelectric element illustrated in FIG. 7 and output sound pressure;

FIG. 16 is a graph illustrating an example of relation between the size of the upper electrode and reception sensitivity;

FIG. 17 is a graph illustrating an example of relation between the size of the upper electrode and the capacitance of the piezoelectric element;

FIG. 18 is a graph illustrating an example of relation between the film thickness of the piezoelectric body and breakdown voltage;

FIG. 19 is a graph illustrating an example of the relationship between the film thickness of the piezoelectric body and the capacitance of the piezoelectric element;

FIG. 20A to 20C are diagrams illustrating another example of the configuration of the piezoelectric body;

FIG. 21 is another example of the configuration of the piezoelectric body;

FIG. 22 is a perspective view illustrating a configuration of the piezoelectric body according to a second embodiment;

FIG. 23 is a cross-sectional view illustrating an example of a cross-sectional structure of the piezoelectric body illustrated in FIG. 22; and

FIG. 24 is a cross-sectional view illustrating another example of a method for manufacturing an electromechanical transducer element according to the present disclosure.

The accompanying drawings are intended to depict embodiments of the present invention and should not be interpreted to limit the scope thereof. The accompanying drawings are not to be considered as drawn to scale unless explicitly noted. Also, identical or similar reference numerals designate identical or similar components throughout the several views.

### DETAILED DESCRIPTION

In describing embodiments illustrated in the drawings, specific terminology is employed for the sake of clarity. However, the disclosure of this specification is not intended to be limited to the specific terminology so selected and it is to be understood that each specific element includes all technical equivalents that have a similar function, operate in a similar manner, and achieve a similar result. As used herein, the singular forms “a,” “an,” and “the” are intended to include the plural forms as well, unless the context clearly indicates otherwise. Components having the same function and configuration are appended with the same reference codes, and redundant descriptions thereof may be omitted. Components in the drawings may be partially omitted or simplified to facilitate understanding of the configurations.

For reducing the size, thickness, and frequency, of an ultrasonic transducer, naturally, each of electromechanical transducer elements to vibrate is preferably as small as possible to be disposed densely. Further, preferably, piezoelectric elements of the electromechanical transducer element are also made as small as possible and disposed densely.

However, as piezoelectric elements become small, sound pressure during vibration and reception sensitivity tend to decrease.

In view of the foregoing, a description is given below of embodiments according to the present disclosure.

An ultrasonic diagnostic apparatus 10 according to the present disclosure includes an ultrasonic probe 1, a display 61, a control panel 62, and a controller 63 that controls the ultrasonic probe 1. The ultrasonic probe 1 applies an ultrasonic wave to a measurement target 9 and detects vibrations of the ultrasonic wave reflected from the measurement target 9. The display 61 visualizes and displays a signal from the ultrasonic probe 1.

As illustrated in FIG. 2, the controller 63 includes an ultrasonic pulse generator 64, a converter 65, and an ultrasonic image forming unit 66. The ultrasonic pulse generator 64 generates a pulsed electric signal for generating an ultrasonic signal. The converter 65 converts an echo signal received from the ultrasonic probe 1 into an electric signal. The ultrasonic image forming unit 66 generates a two-dimensional or three-dimensional ultrasonic image, or various Doppler images from echo signals.

The ultrasonic pulse generator 64 and the converter 65 may be an ultrasonic transmitter-receiver separate from the controller 63, for example.

The display 61 is, for example, a liquid crystal display (LCD) or a monitoring device and displays an image generated by the ultrasonic image forming unit 66.

The control panel 62 is an input device for a user to input parameters and the like so as to appropriately diagnose the measurement target 9. The control panel 62 may include a push button and a touch panel.

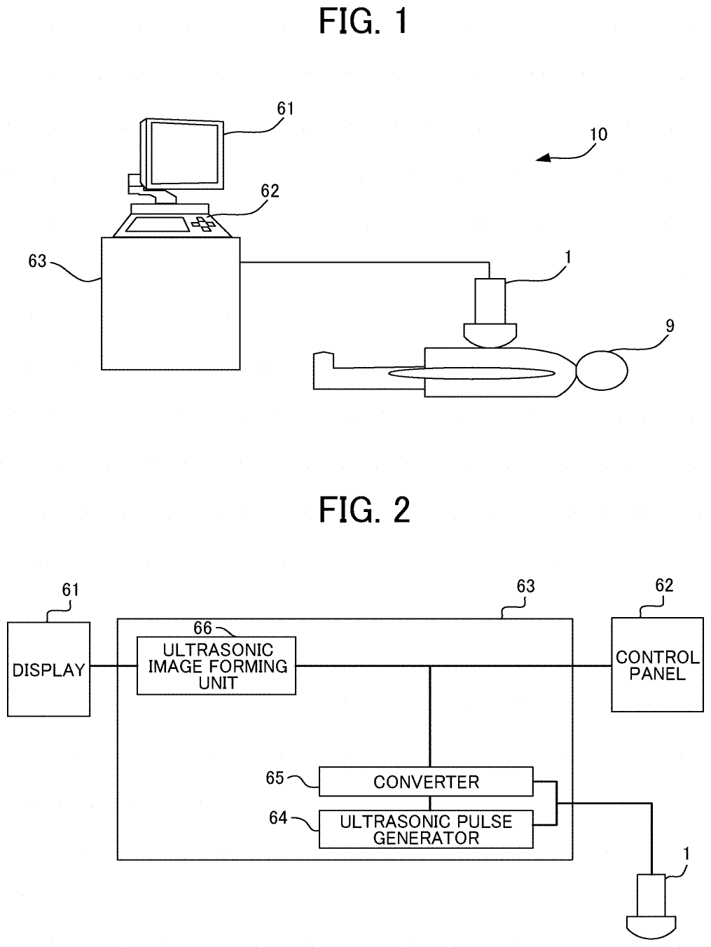

As illustrated in FIG. 1, the ultrasonic probe 1 is electrically connected to the controller 63 via a cable or the like. The ultrasonic probe 1 transmits an ultrasonic signal toward the measurement target 9 which is a human body or an object and receives the ultrasonic signal reflected as an echo from the measurement target 9.

The ultrasonic diagnostic apparatus 10 can visualize an inside of the measurement target 9 and diagnosis the inside by transmitting and receiving an ultrasonic signal.

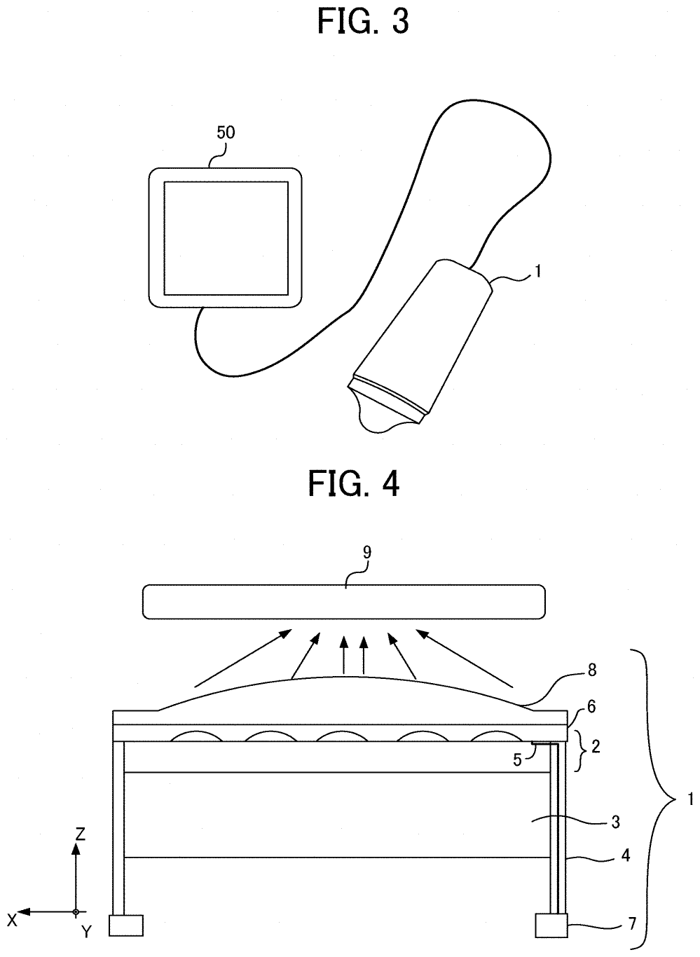

Alternatively, as illustrated in FIG. 3, the diagnosis may be made using a terminal 50 (an information processing terminal) and the ultrasonic probe 1 connected to the terminal 50 by a cable.

As illustrated in FIG. 4, the ultrasonic probe 1 includes a support board 3, a piezoelectric micro-machined ultrasonic transducer (PMUT) chip 2 which is an ultrasonic transducer disposed on the support board 3, a flexible printed board 4, wiring 5, a connector 7, and an acoustic lens 8.

The PMUT chip 2 is connected to the flexible printed board 4 via the wiring 5 and is connected from the connector 7 to the controller 63 via a circuit board.

The support board 3 functions as a backing plate to support the PMUT chip 2.

The acoustic lens 8 is made of silicon resin and used for focusing the ultrasonic wave transmitted from the PMUT chip 2 on the measurement position of the measurement target 9.

The acoustic lens 8 has a so-called dome shape in which the center portion is thicker than the peripheral portion. The acoustic lens 8 tightly contacts the measurement target 9 and deflects ultrasonic wave in a pseudo manner due to the difference in thickness between the center portion and the peripheral portion, thereby focusing the ultrasonic wave. The acoustic lens 8 has a function of focusing ultrasonic wave in at least one direction and does not necessarily focus the ultrasonic wave to one point.

The acoustic lens 8 and the PMUT chip 2 are bonded to each other by adhesive 6.

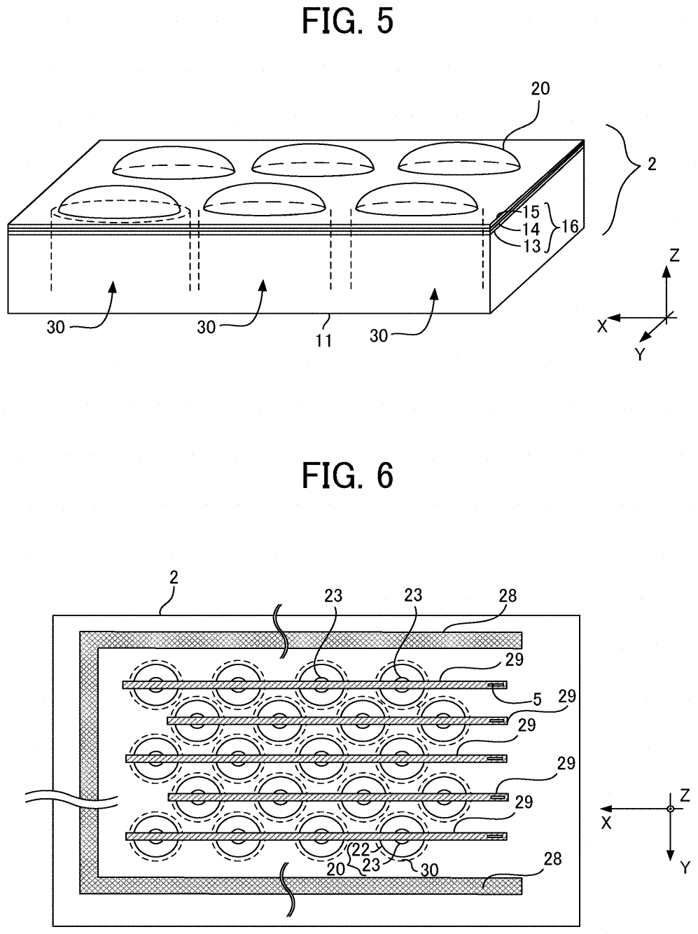

As illustrated in FIG. 5, the PMUT chip 2 includes a silicon substrate 11, an oxide film 13 disposed on the silicon substrate 11, a silicon layer 14, and an oxide film 15.

The oxide film 13, the silicon layer 14, and the oxide film 15 together function as a diaphragm 16 by application of a voltage to a piezoelectric element 20 as described later.

In the present embodiment, a plurality of piezoelectric elements 20 is disposed on the upper side of the diaphragm 16. Each piezoelectric element 20 has a so-called dome-shape in which the center portion is thicker than the peripheral portion.

As indicated by broken lines, void areas 30 are secured on the side of the diaphragm 16 opposite the piezoelectric elements 20, that is, on the lower side of the diaphragm 16 in FIG. 5.

In the description with reference to FIG. 5 and subsequent drawings, the direction perpendicular to the surface of the silicon substrate 11 is referred to as a Z axis direction, the arrangement direction of the piezoelectric elements 20 on the silicon substrate 11 is referred to as an X axis direction, and the direction perpendicular to the X axis and the Z axis is referred to as a Y-axis direction.

When viewed from the upper side (downstream side in the Z axis direction or +Z side) in the drawing, as illustrated in FIG. 6, in PMUT chip 2, the plurality of piezoelectric elements 20 is disposed in an array, and a signal line 29 electrically connects a row of piezoelectric elements 20. The signal line 29 is connected to the wiring 5 at the end of the PMUT chip 2 as illustrated in FIG. 3.

The signal line 29 extends in the X axis direction and is connected to an upper electrode 23, which will be described later.

As illustrated in FIG. 6, a ground (GND) line 28, having a reference potential, is disposed around the piezoelectric elements 20 on the PMUT chip 2. The ground line 28 is connected to a lower electrode 21 described later.

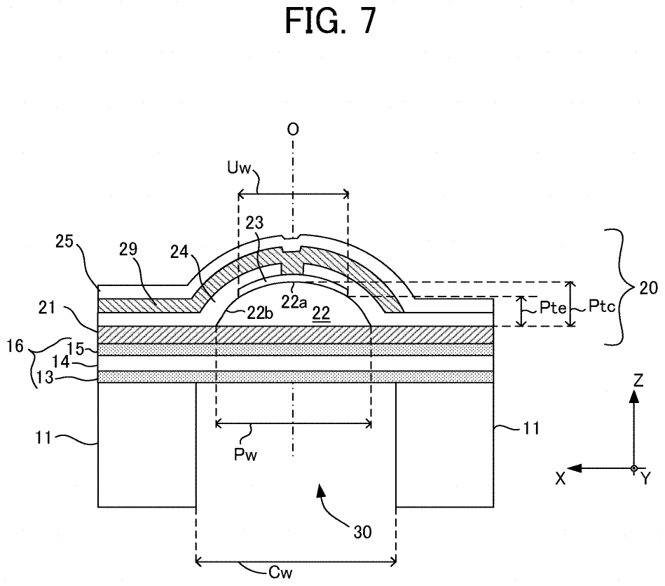

The void area 30 is a columnar opening in the silicon substrate 11. As illustrated in FIG. 6 and the cross-sectional view in FIG. 7, the oxide film 13 on the upper side (+Z side) of the void area 30 serves as an end wall (upper bottom wall) of the void area 30 opposite the open side of the void area.

Note that FIG. 7 illustrates a cross-sectional view when the piezoelectric element 20 and the silicon substrate 11 are cut on a plane including a center axis O of one piezoelectric element 20 and the X axis, and the void area 30 has a width Cw in the X direction.

Further, in the present embodiment, as illustrated in FIG. 7, the piezoelectric element 20 includes the lower electrode 21, as a first electrode, on the diaphragm 16, a dome-shaped piezoelectric body 22, and the upper electrode 23, as a second electrode, on the upper face of the piezoelectric body 22.

Further, the piezoelectric element 20 includes an insulation layer 24 above the upper electrode 23, the signal line 29, and a protective layer 25 for protecting the signal line 29.

In the present embodiment, the piezoelectric body 22 is dome-shaped, but the shape is not limited thereto.

The lower electrode 21 is a platinum (Pt) layer in the present embodiment, but the material is not limited thereto, and a conductive metal material or the like can be used.

The upper electrode 23 is also a platinum (Pt) layer, but the material is not limited thereto, and a conductive metal material or the like can be used. Desirably, the upper electrode 23 and the lower electrode 21 are made of the same material, but different materials may be used.

The piezoelectric body 22 is a piezoelectric member made of lead zirconate titanate (PZT) in the present embodiment. The piezoelectric body 22 has a dome shape in which a center portion 22a is thicker than a peripheral portion 22b.

The piezoelectric body 22 is mechanically deformed by application of a drive voltage between the upper electrode 23 and the lower electrode 21. By causing periodic fluctuations in the drive voltage, a vibration of a predetermined frequency can be generated. As a result, the diaphragm 16 in contact therewith is vibrated, generating ultrasonic wave.

Further, as such an ultrasonic wave vibrates the piezoelectric body 22, the piezoelectric body 22 is polarized to generate a potential difference between the upper electrode 23 and the lower electrode 21. Thus, the piezoelectric body 22 also functions as a detector to detect the vibration as an electric signal.

As described above, in the present embodiment, the PMUT chip 2 functions as an electromechanical transducer element that periodically expands and contracts the piezoelectric body 22 by a potential difference between the upper electrode 23 and the lower electrode 21, that is, an electric signal, to generate vibration. In particular in the present embodiment, the PMUT chip 2 functions as an ultrasonic transducer that generates a sound wave in an ultrasonic range with such vibrations.

The insulation layer 24 is for preventing a short circuit between the upper electrode 23 and the lower electrode 21 and a short circuit between the signal line 29 and the lower electrode 21.

A description is given below of, as the first embodiment of the present disclosure, an example of a method for manufacturing the PMUT chip 2 having the piezoelectric elements 20 described above.

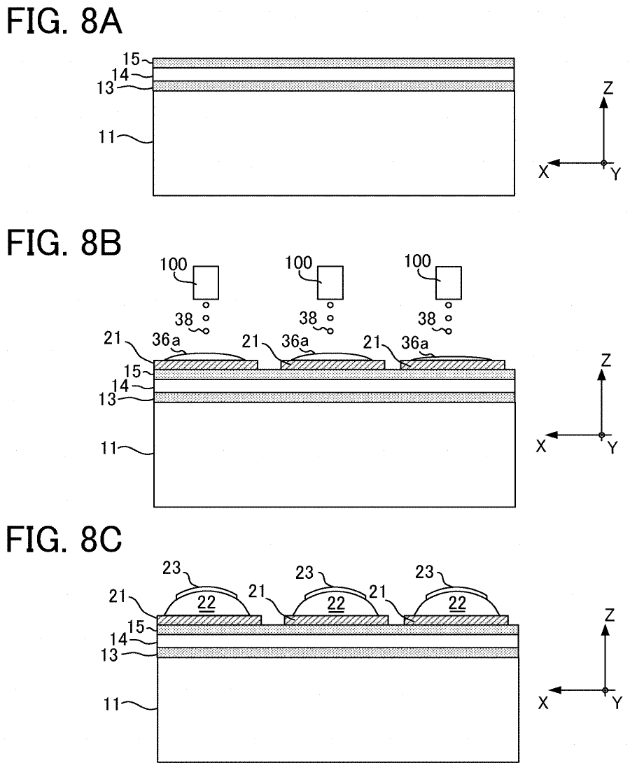

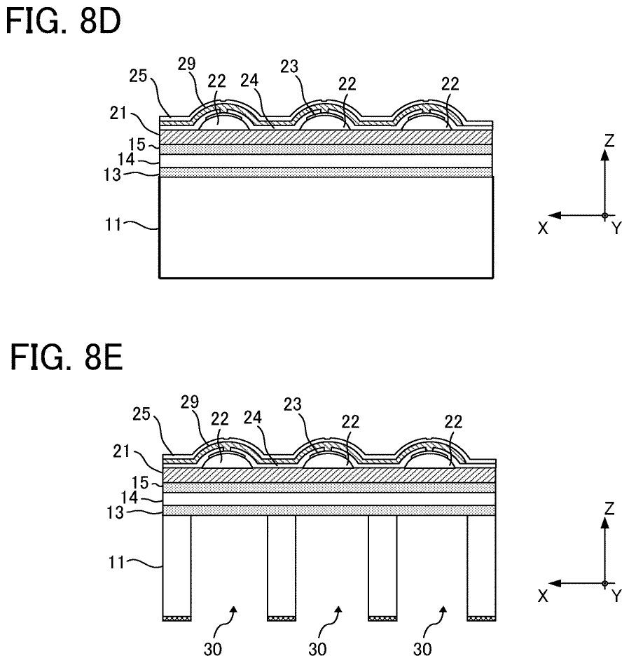

As illustrated in FIG. 8A, the oxide film

having a thickness of about 50 nm to 1000 nm is formed on the silicon substrate 11 (a general silicon substrate), as an oxide film called a buried oxide (BOX) layer for silicon on insulator (SOI).

Next, the silicon layer 14 having a thickness not greater than 5 μm is formed as active SOI layer, and the oxide film 15 having a thickness of about 50 nm to 1000 nm is formed thereon, as an insulation layer. Described above are processes for forming a substrate. That is, the silicon substrate 11, the oxide film, the silicon layer 14, and the oxide film 15 together form a base substrate. A typical method for manufacturing an SOI wafer may be used.

Next, as illustrated in FIG. 8B, the lower electrode 21 which is the first electrode is formed.

A titanium dioxide (TiO₂) layer having a thickness of about 50 to 200 nm may be formed as a tight contact layer between the lower electrode 21 and the oxide film 15 that is a base.

As an example of the method for manufacturing the titanium dioxide (TiO₂) layer, a titanium film of 30 to 200 nm is formed by a sputtering, and then oxidization is caused by rapid thermal anneal (RTA) in an oxygen atmosphere.

As the lower electrode 21, a platinum (Pt) film of 50 to 500 nm is formed by, for example, sputtering. Described above is a process for forming the first electrode.

As illustrated in FIG. 8B, the lead zirconate titanate (PZT) film is formed on the lower electrode 21 by a so-called chemical solution deposition (CSD) method. Specifically, the PZT film is formed in the steps of spin coat of precursor fluid; drying; thermal decomposition; and crystallization. The starting materials of the precursor fluid include lead acetate, a zirconium alkoxide compound, and a titanium alkoxide compound.

At this time, liquid discharge heads 100 selectively apply CSD droplets 38 to a piezoelectric layer 36a, which becomes the piezoelectric body 22.

The liquid discharge head 100 is an inkjet head capable of applying the CSD droplets 38 to a given position on the silicon substrate 11 while the silicon substrate 11 or the liquid discharge head 100 moves.

At this time, surface treatment is performed so that the surface of the portion forming the piezoelectric layer 36a is made hydrophilic and the surface around the portion forming the piezoelectric layer 36a is made water-repellent.

When the CSD droplets 38 are applied to the substrate having such a surface property, even when the landing positions of the CSD droplets 38 vary due to minute position errors of the liquid discharge heads 100, the CSD droplets 38 are selectively applied only to the hydrophilic portions, and the CSD droplet 38 are not applied to the water-repellent portions.

After the CSD droplets 38 are applied to the desired positions in this way, the partially applied CSD droplets 38 are dried, thermally decomposed, and crystallized.

There is a concern that cracks are likely to occur when the thickness of film formed by application of one time is large. Therefore, in the present embodiment, the CSD droplets 38 applied by the liquid discharge head 100 are adjusted so that the film thickness after crystallization is within about 100 nm.

After the crystallization is completed, the liquid discharge heads 100 repeatedly apply the CSD droplets 38 until the piezoelectric body 22 has a desired thickness. Described above are processes for forming a piezoelectric body.

In the present embodiment, the piezoelectric body 22 of 1 μm to 4 μm is formed by repeating the above-described application 10 to 40 times.

At this time, the viscosity and drying speed of the CSD droplets 38 can be controlled. For example, as the viscosity increases, the curvature of the surface of the piezoelectric body 22 after the film formation increases.

By controlling the physical properties of the CSD droplets 38 as a coating material, the thickness of the center portion and the peripheral portion of the piezoelectric body 22 can be controlled.

Further, as illustrated in FIG. 8C, the platinum upper electrode 23 having a thickness of 50 to 300 nm is formed on the +Z side of the piezoelectric body 22 by sputtering or the like. Described above are processes for forming a second electrode.

At this time, desirably, the upper electrode 23 is formed in conformity to the upper surface of the dome-shaped piezoelectric body 22, and the diameter of the upper electrode 23 is smaller than the outer diameter of the piezoelectric body 22. In particular, in the case of the dome-shaped piezoelectric body 22, when the diameter of the upper electrode 23 is smaller than the outer diameter of the piezoelectric body 22, a short circuit can be prevented between the upper electrode 23 and the lower electrode 21.

As the upper electrode 23, a predetermined pattern is formed by photolithography etching.

Next, as illustrated in FIG. 8D, the insulation layer 24 is formed as an insulation film on the upper electrode 23. In the present embodiment, the insulation layer 24 is a silicon oxide film of 0.4 to 1.0 μm formed by a plasma-enhanced chemical vapor deposition (CVD), from monosilane (SiH₄) and nitrous oxide (N₂O) gas as raw materials.

Then, the signal line 29 is formed through processes of opening of a contact hole 41 in the insulation layer 24 by photolithography etching, formation of an aluminum-copper (Al—Cu) film of 1 μm and a titanium (Ti) film of 50 nm as wiring materials by sputtering, and patterning by photolithography etching.

Further, a silicon nitride (Si₃N₄) film is formed as the protective layer 25, and an opening is formed only in portions of electrode terminals connected to the wiring 5.

In the present embodiment, the protective layer 25 is a silicon nitride (Si₃N₄) film of 0.5 to 1.5 μm deposited by a plasma CVD method, from monosilane (SiH₄) and nitrous oxide ammonia (NH₃) gas as raw materials.

The openings of the electrode terminal portions are formed by photolithography etching.

As illustrated in FIG. 8E, the void area 30 is present on the side of the silicon substrate 11 opposite the piezoelectric body 22, that is, on the back side of the silicon substrate 11.

Specifically, after the thickness of the silicon substrate 11 is adjusted to about 20 μm to 200 μm by back grinding and polishing, the surface (i.e., the front side or +Z side surface) of the silicon substrate 11 provided with the piezoelectric elements 20 is attached to the support board.

A resist mask having a desired pattern is pattered by photolithography on the back side (−Z side surface) of the silicon substrate 11, and the silicon substrate 11 is etched. The etching can be easily performed using a silicon deep etcher by a so-called Bosch process (alternately repeating etching with SF₆ plasma and deposition of a side wall protective film with C₄F₈ plasma).

At this time, the portion of the silicon substrate 11 free of the resist mask is dug by etching to expose the oxide film, thereby forming the void areas 30. Described above are processes for forming a void area (the void area 30) on the side of the silicon substrate 11 opposite the piezoelectric body 22.

Then, the support wafer is separated, and dicing and the like are performed. Thus, manufacturing of a wafer of the PMUT chip 2 completes.

To use the PMUT chip 2 manufactured through such a manufacturing processes as the ultrasonic transducer of the ultrasonic probe 1, it is preferred to secure the sound pressure, reduce the size and the thickness, and increase the frequency.

However, reducing the size and increasing the frequency of piezoelectric elements generally result in a decrease in sound pressure due to a decrease in the amplitude of the vibrating portion.

However, according to the research by the inventors, high frequency, high resolution, appropriate sound pressure, and reception sensitivity can be secured by controlling the size of the piezoelectric body 22, the size of the upper electrode 23, and the size of the void area 30, as described in detail below.

Referring to FIG. 7, the void area 30 has the width Cw on the cross section of the piezoelectric element 20 and the silicon substrate 11.

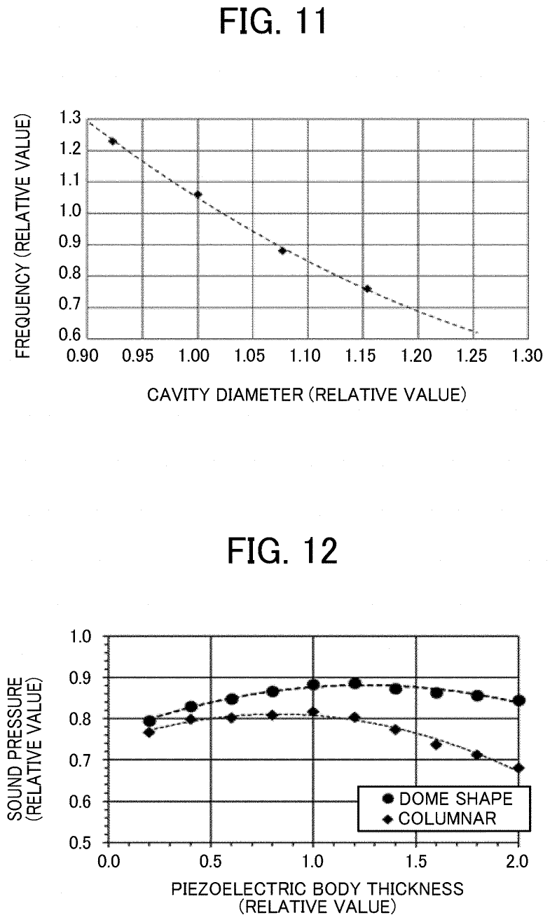

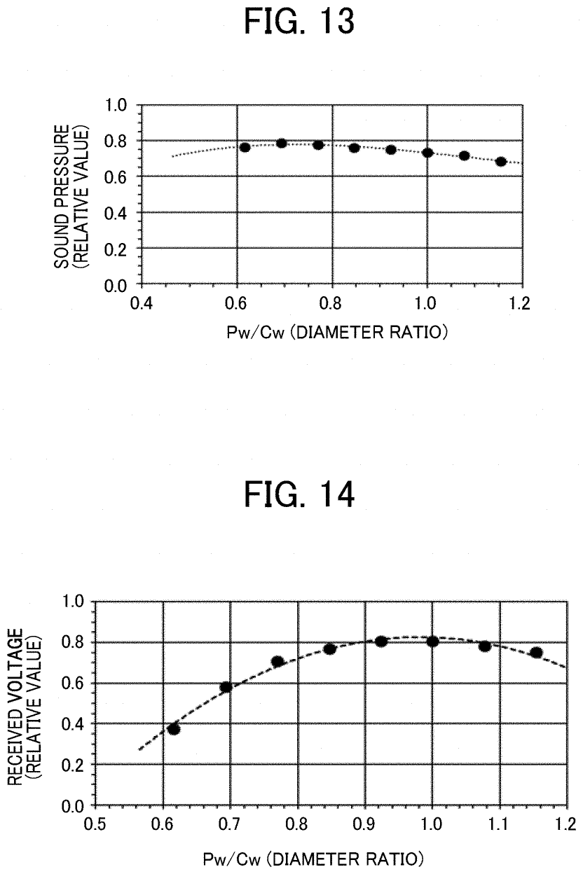

The “width Cw of the void area 30” is, for example, the width of the void area 30 when the piezoelectric body 22 is viewed along the Z direction, which is the layer direction, and may be the width of the void area 30 as viewed on the cross section including the center axis (the center axis O of one piezoelectric element 20) of the piezoelectric body 22. The width Cw may be in the X direction, the Y direction, or any other direction. In the present embodiment, since the void area 30 is columnar, the width Cw of the void area 30 is equal in any radial direction of the void area 30. Therefore, the width Cw is referred to as a cavity diameter in FIG. 11.

Referring to FIG. 7, since the piezoelectric body 22 has a circular dome shape when viewed from above, the piezoelectric body 22 has a width Pw (PZT diameter), the center portion 22a of the piezoelectric body 22 has a thickness Ptc (a thickness at the center of the piezoelectric body 22), and the portion of the piezoelectric body 22 at an end of the upper electrode 23 has a thickness Ptc. As described above, the “width Pw of the piezoelectric body 22” is the width of the piezoelectric body 22 on the cross section cut along the layer direction of the piezoelectric elements 20. In FIG. 7, the width Pw is indicated in the cross-sectional view for simplicity. In FIG. 7, the upper electrode 23 has a width Uw, and the piezoelectric body 22 has a thickness Pte at the end of the upper electrode 23.

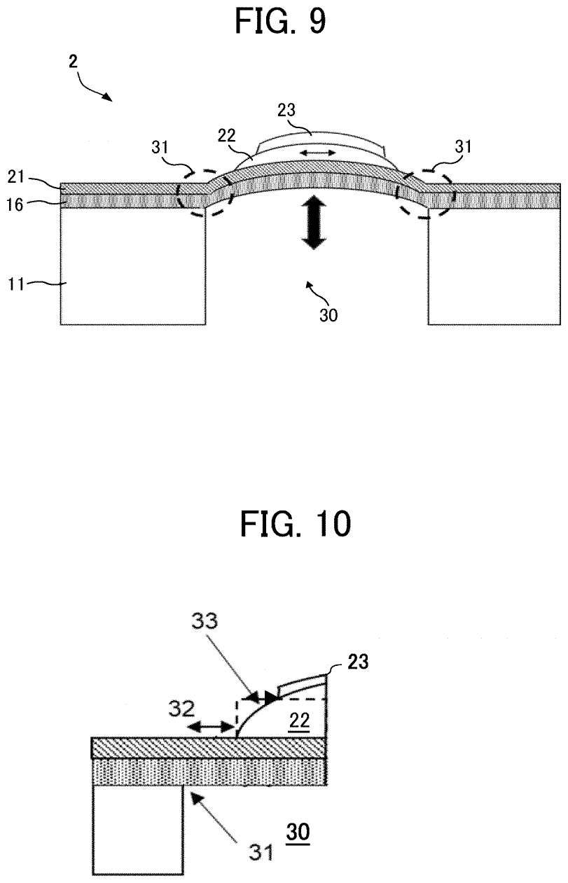

FIG. 9 illustrates a state in which the ultrasonic transducer (the PMUT chip 2) vibrates. The upper wiring and insulation film are omitted for simplification of the explanation. As an electric potential is applied to the lower electrode 21 and the upper electrode 23 of the piezoelectric body 22 at a high frequency, the piezoelectric body 22 expands and contracts in the horizontal direction. As a result, the piezoelectric element 20 and the diaphragm 16 vibrate in the vertical direction. This vibration is output as ultrasonic wave. Since the piezoelectric element 20 is fixed to the silicon substrate 11, the piezoelectric element 20 vibrates at an inflection point 31 as a boundary. In the case where the energy of the vibration is the same, when the area around the inflection point 31 is soft and light, the amount of displacement is large, and as a result, the sound pressure and reception voltage increase. Therefore, preferably, no hard and heavy member, such as the piezoelectric body 22, is disposed in the vicinity of the inflection point 31. FIG. 10 is an enlarged view of the vicinity of the inflection point 31 illustrated in FIG. 9. A space 32 (a distance) between an end of the void area 30 and an end of the piezoelectric body 22, in the direction perpendicular to the vibration direction, has a width represented as (Cw−Pw)/2, using the width Cw and the width Pw illustrated in FIG. 7. A certain distance of the space 32 is required in consideration of deviations of the pattern in manufacturing of the piezoelectric element. A space 33 (a distance) between the end of the piezoelectric body 22 and an end of the upper electrode 23, in the direction perpendicular to the vibration direction, has a width represented as (Pw−Uw)/2. The portion expressed as the space 33, without the upper electrode 23, do not affect the piezoelectric characteristics and, such portions are desirably thin and narrow.

As illustrated in FIG. 11, increasing the width Cw (the cavity diameter) of the void area 30 results in decreases in the frequency of ultrasonic wave generated by the resonance of the diaphragm 16.

Specifically, in order to cause resonance in the frequency range of 5 MHz to 20 MHz, the cavity diameter is about 30 μm to 100 μm.

In the present embodiment, the frequency is 10 MHz, and the width Cw of the void area 30 is within the range of 60 μm to 70 μm.

Next, the shape of the piezoelectric body 22 is described below.

In the present embodiment, the piezoelectric body 22 has a dome shape in which the center portion 22a is thicker than the peripheral portion 22b as described above with reference to FIG. 8B.

When the piezoelectric body 22 has a columnar shape, the bendability of the piezoelectric body 22 is uniform. By contrast, when the piezoelectric body 22 has a dome shape, the peripheral portion 22b is relatively easily displaced because the thickness is smaller. As a result, a high sound pressure and a high reception sensitivity can be maintained in the case of the dome shape.

FIG. 12 is a graph illustrating relative evaluation of sound pressure depending on the shape of the piezoelectric body 22. In FIG. 12, the vertical axis represents the sound pressure value that are relative values in arbitrary unit, that is, when a predetermined sound pressure is 1.

Further, FIG. 12 is a graph illustrating the magnitude of the sound pressure detected when ultrasonic wave is emitted from the PMUT chip 2 serving as an ultrasonic transducer.

Referring to FIG. 7, the center portion 22a of the piezoelectric body 22 has the thickness Ptc. As is clear from FIG. 12, when the thickness of the piezoelectric body 22 is the same between the columnar shape and the dome shape, the dome shape can attain a higher sound pressure. Accordingly, preferably, the piezoelectric body 22 has a dome shape.

Further, in the case where the piezoelectric body 22 has a columnar shape, the sound pressure decreases as the thickness Ptc of the center portion 22a of the piezoelectric body 22 increases due to the weight thereof. By contrast, in the case of the dome shape, even when the thickness Ptc of the center portion 22a of the piezoelectric body 22 is increased, decreases in sound pressure can be inhibited.

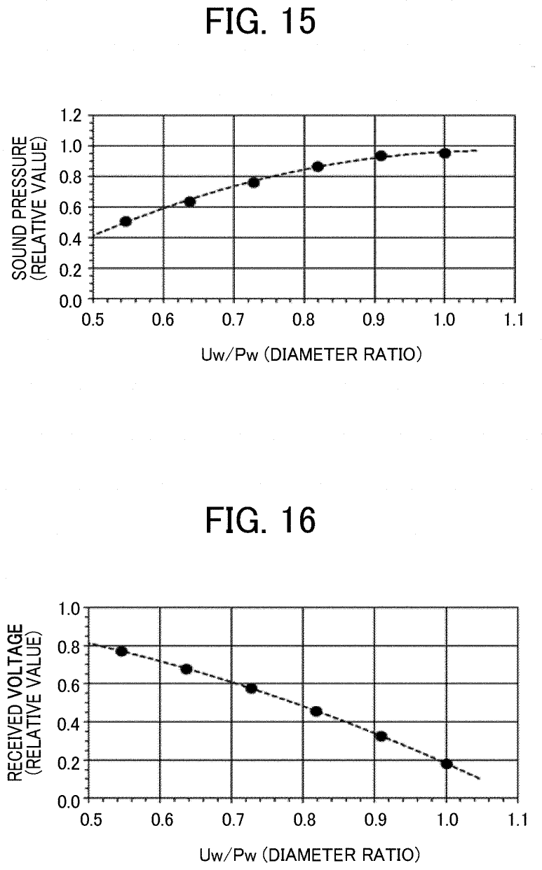

FIG. 15 illustrates the relationship between the PZT diameter and the transmitted sound pressure. FIG. 14 illustrates the relationship between the PZT diameter and the received voltage.

Therefore, in the present embodiment, the piezoelectric element 20 satisfies Expression 1 below, where Cw represents the width of the void area 30 (the cavity diameter) and Pw represents the width of the piezoelectric body 22 corresponding to the diameter (PZT diameter) of the piezoelectric body 22.

0.65≤Pw/Cw≤0.95 Equation 1

Expression 1 defines the size of the piezoelectric body 22 relative to the size of the void area 30. When the ratio of Pw/Cw is smaller than the lower limit of Expression 1, the piezoelectric body 22 is too small relative to the void area 30, and the diaphragm 16 does not sufficiently vibrate, resulting in a decrease in sound pressure.

On the other hand, when the value Pw/Cw is larger than the upper limit of Expression 1, the piezoelectric body 22 is too large relative to the void area 30. Accordingly, as illustrated in FIGS. 9 and 10, since the piezoelectric body 22 is heavy and hard, the diaphragm 16 does not vibrate sufficiently, resulting in a decrease in sound pressure.

As is clear from FIG. 14, for the same reason, from the viewpoint of the reception sensitivity, the width Pw of the piezoelectric body 22 and the width Cw of the void area 30 are set within the range of Expression 1. As a result, a relative sound pressure value of 0.5 or greater can be secured. Further, satisfying 0.7≤Pw/Cw≤0.9 within the range of Expression 1 is more preferable because the relative sound pressure can increase further.

Further, the piezoelectric element 20 satisfies Expression 2, where Uw represents the width of the upper electrode 23 (see FIG. 7).

0.6≤Uw/Pw≤0.9 Expression 2

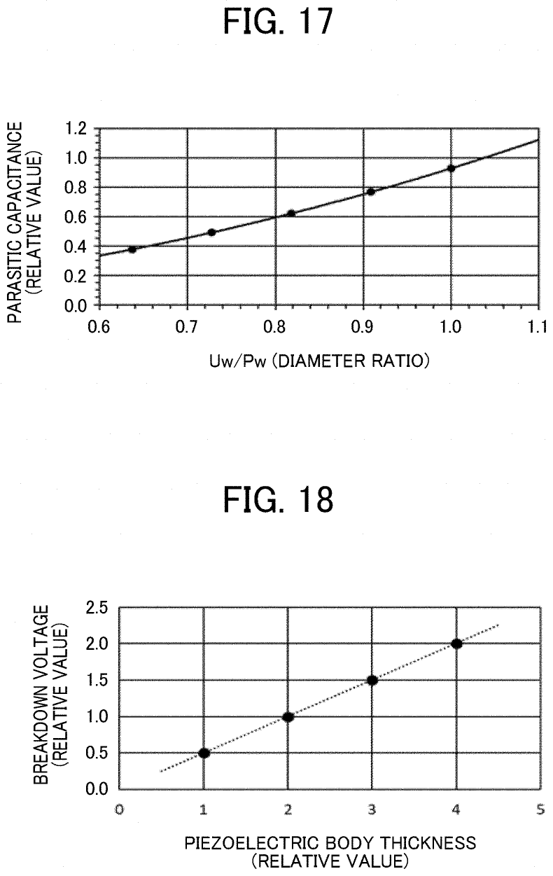

Expression 2 defines the size of the upper electrode 23 relative to the piezoelectric body 22. FIG. 15 is a graph illustrating the relationship between Expression 2 and the relative sound pressure value. As is clear from FIG. 15, as the width Uw becomes larger relative to the width Pw, the piezoelectric effect increases, and the sound pressure improves. However, as illustrated in FIG. 16, the received voltage decreases as the width Uw of the upper electrode 23 increases relative to the width Pw of piezoelectric body 22. FIG. 17 illustrates the relationship between the width Uw of the upper electrode 23 and the capacitance of the piezoelectric element 20. As illustrated in FIG. 17, as the upper electrode 23 becomes larger, the capacitance of the piezoelectric body 22, which is sandwiched between the upper electrode 23 and the lower electrode 21, increases.

When the capacitance of the piezoelectric element 20 becomes large, an unintended capacitance component is placed on the circuit, which decreases the responsiveness in a high frequency band and decreases the voltage.

In the present embodiment, the plurality of piezoelectric elements 20 is arranged in the PMUT chip 2, each piezoelectric element 20 includes the upper electrode 23 and the lower electrode 21, and the lower electrode 21 is shared in the array direction. That is, since the capacitance of the piezoelectric element 20 becomes a combined capacitance of parallel connection, the influence of signal delay and voltage drop is large. Accordingly, it is preferred to minimize the capacitance of the piezoelectric element 20 per one piezoelectric element 20. Therefore, determining an optimum value in consideration with the results illustrated in FIGS. 15, 16, and 17 is important.

Therefore, according to the present embodiment, the piezoelectric element 20 satisfies Expression 2 to reduce the capacitance of the piezoelectric element 20. Further, the width Pw of the piezoelectric body 22 and the width Uw of the upper electrode 23 are set within the range defined by Expression 2. With this configuration, while securing the relative sound pressure value of 0.5 or greater, the reception voltage can be secured and the capacity of the piezoelectric element 20 can be reduced, thereby inhibiting decreases in responsiveness to high frequency. Further, satisfying 0.7≤Uw/Pw≤0.8 within the range of Expression 2 is more preferable because both the responsiveness and the sound pressure can be secured at the same time.

Further, the piezoelectric element 20 satisfies Expression 3, where Pte represents the thickness of the piezoelectric body 22 at the end of the upper electrode 23, and Ptc represents the thickness of the center portion 22a of the piezoelectric body 22.

0.2≤Pte/Ptc≤1.0 Expression 3

In general, the dome-shaped piezoelectric body shape is approximated by a mathematical formula such as Y=−ax2+b as described in U.S. Pat. No. 9,533,502-B2. FIG. 18 illustrates the relationship between the film thickness of the piezoelectric body 22 and the breakdown voltage.

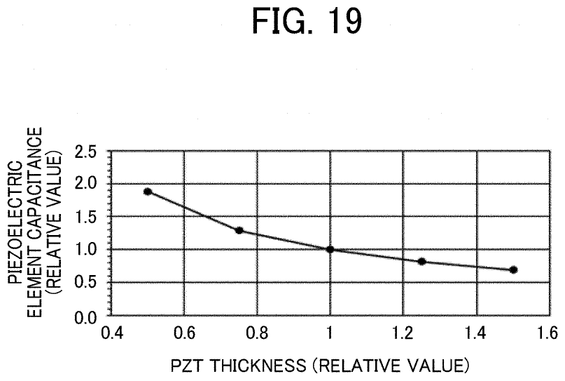

The thickness Pte of the piezoelectric body 22 at the end of the upper electrode 23 is a typical value among the thicknesses of the peripheral portion 22b. When the thickness Pte is extremely thin, dielectric breakdown occurs between the upper electrode 23 and the lower electrode 21. When the thickness Pte is zero (0), short circuit occurs. By contrast, when the thickness Pte is extremely thick, the thickness Pte becomes close to the thickness Ptc of the center portion 22a in the case of the dome shape. Then, the width Uw of the upper electrode 23 becomes small, and Expression 2 is not satisfied. FIG. 19 illustrates the relationship between the thickness of the piezoelectric body 22 and the capacitance of the piezoelectric element 20. As is clear from FIG. 19, the capacitance of the piezoelectric element 20 can be reduced by increasing the thickness of the piezoelectric body 22.

Therefore, when the piezoelectric element 20 satisfies Expression 3, the breakdown voltage between the upper electrode 23 and the lower electrode 21 can be secured, the sound pressure and the received voltage can improve, and the piezoelectric element capacitance can be optimized. Further, in the present embodiment, as described above, the width Uw of the upper electrode 23 and the width Pw of the piezoelectric body 22 are set within the range defined by Expression 2. Therefore, in the present embodiment, in addition to Expression 3, preferably, the thickness Pte of the piezoelectric body 22 at the end of the upper electrode 23 is equal to or smaller than 3 μm (Pte≤3 μm).

Further, in the range defined by Expression 3, satisfying 1 μm≤Pte≤2 μm is more preferable because strength against dielectric breakdown and improvement of sound pressure due to the dome shape can be achieved at the same time.

In the present embodiment, the thickness Ptc at the center portion 22a of the piezoelectric body 22 satisfies Expression 4.

1 μm≤Ptc≤4 μm Expression 4

When the thickness Ptc is larger than the upper limit of Expression 4, the film thickness of the piezoelectric body 22 becomes too large, so that cracks and the like are likely to occur.

By contrast, when the thickness Ptc is smaller than the lower limit of Expression 4, the capacity of the piezoelectric element 20 increases as described above. Also, the dielectric strength decreases, which is not preferable.

Further, within the range of Expression 4, setting the thickness Ptc in range of 1.5 μm≤Ptc≤3 μm is more preferable because the durability improves due to inhibition of cracks, and improvement of sound pressure due to the dome shape can be achieved at the same time.

The description above concerns the optimization of the size of the representative portion of the piezoelectric element 20 according to the present embodiment, but the sizes described above are examples, and embodiments of the present disclosure are not limited thereto.

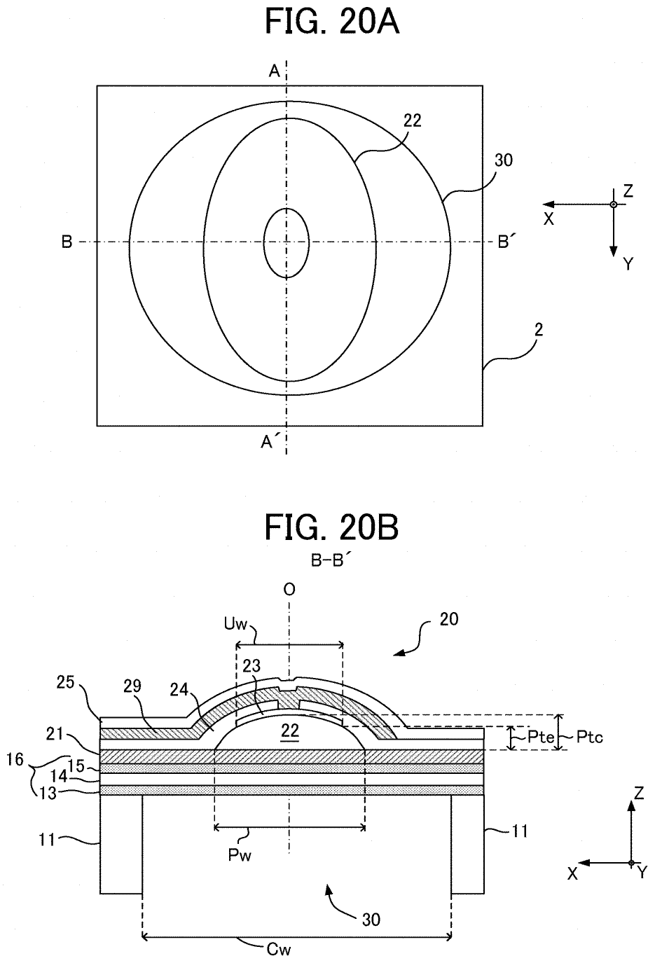

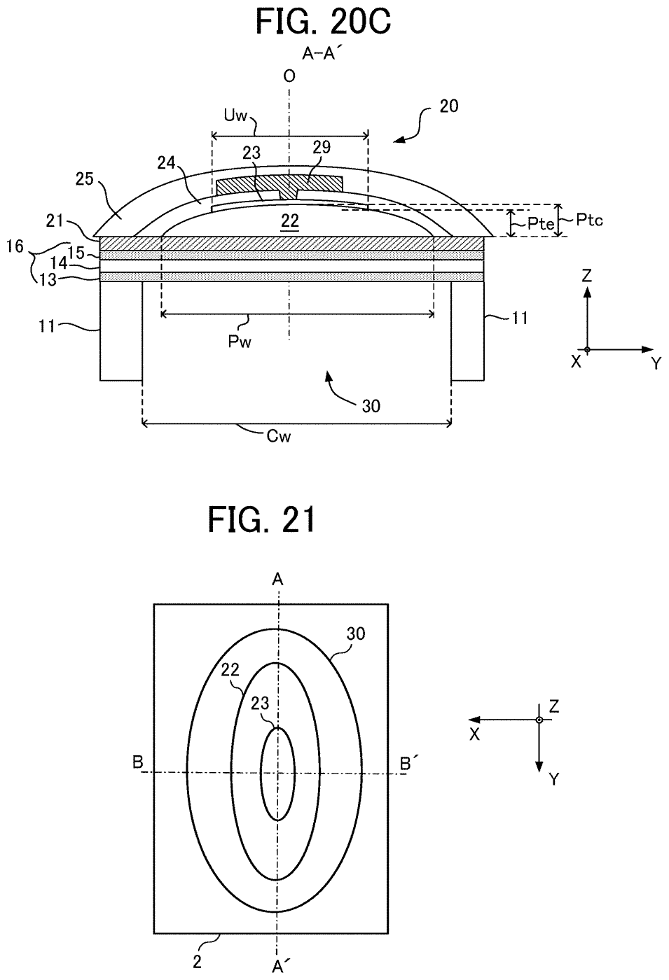

For example, in the present embodiment, the piezoelectric body 22 has a dome shape that is axisymmetric with respect to the center axis, and Expression 5 is satisfied at any position. By contrast, as illustrated in FIG. 20A, when the piezoelectric body 22 has a dome shape that is elliptical when viewed from the +Z direction side, for example, the piezoelectric body 22 may be shaped to satisfy Expression 1 in both the A-A′ cross section and the B-B′ cross section orthogonal to the A-A′ cross section. Further, as illustrated in FIG. 21, in addition to the piezoelectric body 22, also the void area 30 may have an elliptical shape.

FIGS. 20B and 20C illustrate examples of a cross-sectional view of the piezoelectric body 22 having such shapes.

A description is given below of a second embodiment of the present disclosure, in which the piezoelectric element 20 includes a piezoelectric body 22′ having a columnar shape instead of a dome shape.

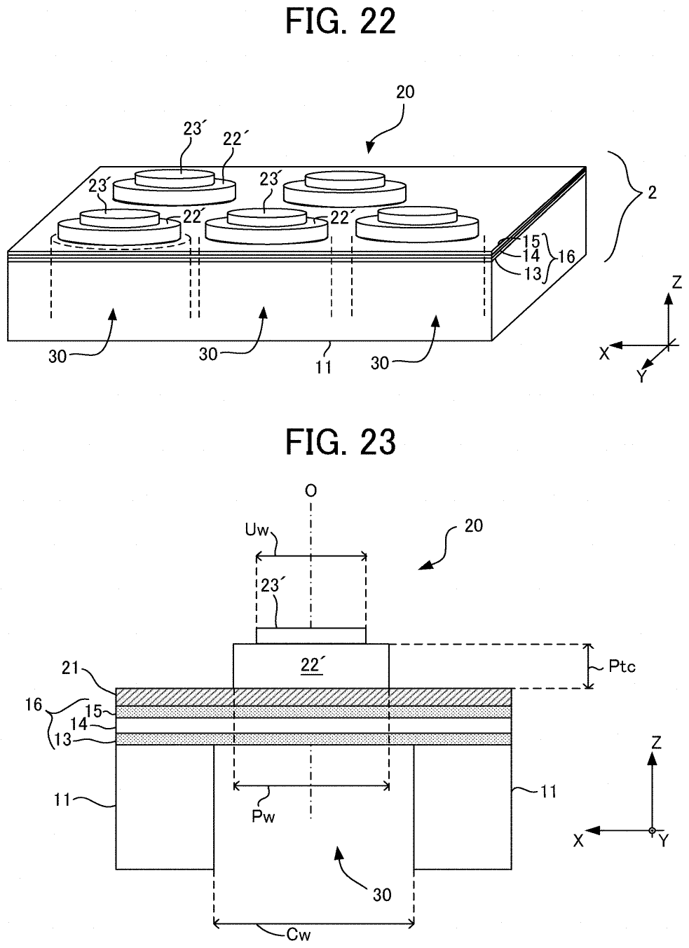

As illustrated in FIG. 22, in the present embodiment, the piezoelectric body 22′ is columnar and the thickness thereof is not different between the center portion and the peripheral portion. The elements same as those of the first embodiment are given the same reference numerals, and redundant descriptions are avoided. Further, the description of portions, such as the insulation layer 24, the protective layer 25, and the signal line 29, which are not different even when the piezoelectric body 22′ is columnar are omitted.

Also in the piezoelectric body 22′, the void area 30 has the width Cw and the piezoelectric body 22′ has the width Pw, on a cross section when the piezoelectric element 20 and the silicon substrate 11 are cut in a plane including the center of the piezoelectric element 20, as illustrated in FIG. 23.

Specifically, the diameter of the columnar piezoelectric body 22′ when viewed from the +Z direction is the width Pw of the piezoelectric body 22′, and, similarly, the diameter of the void area 30 is the width Cw.

Further, the height of the piezoelectric body 22′ in the +Z direction is the thickness Pt of the piezoelectric body 22′. Needless to say, Pt=Pte=Ptc in the present embodiment.

An upper electrode 23′, which is a platinum (Pt) electrode formed on the piezoelectric body 22′, is also columnar.

At this time, the width Uw of the upper electrode 23′ in the cross section of the piezoelectric element 20 is defined as illustrated in FIG. 23.

Also, in the second embodiment, the PMUT chip 2 satisfies Expression 1 and Expression 2, where Uw represents the width of the upper electrode 23′ in the cross section of the piezoelectric element 20.

With such setting, even in the configuration illustrated in FIGS. 22 to 23, high frequency, high resolution, appropriate sound pressure, and reception sensitivity can be secured.

The columnar piezoelectric body 22′ can be manufactured, not inkjet printing, but through the following method. Upon sputtering, CVD, spin coating of sol-gel solution, piezoelectric body 22′ is formed in the thickness of 1 to 4 μm, and patterning is performed by a photolithography-etching method.

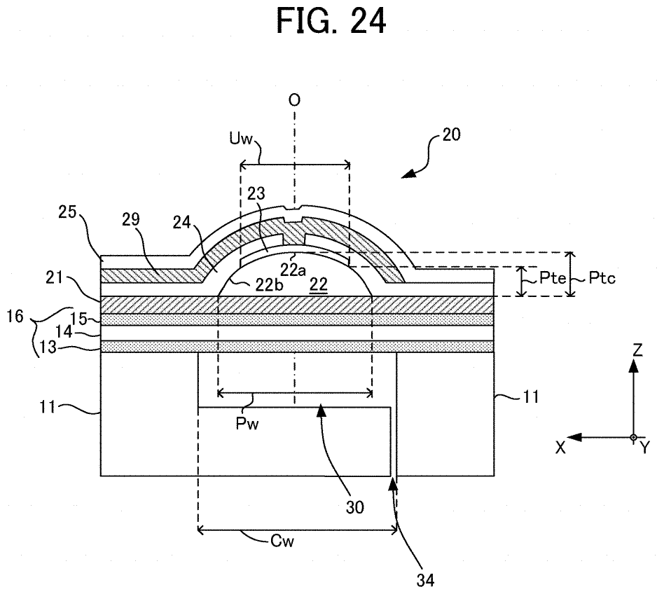

In the first and second embodiments described above, the void area 30 is formed from the back side after the piezoelectric element 20 is formed on the silicon substrate 11 as illustrated, as an example, in FIG. 8E. Alternatively, in the step of forming the SOI on the silicon substrate 11, as illustrated in FIG. 24, silicon may be etched from the surface of the substrate by about 1 to 5 μm in advance. Yet alternatively, as so-called sacrificial etching, a hole 34 (an opening) may be formed in a portion of the wall face of the void area 30, and the silicon substrate 11 may be dry-etched from the hole 34.

Further, the shape of the void area 30 is not limited to the columnar shape but can be changed variously according to the shape of the piezoelectric element 20.

Although the example embodiments are described above, the present disclosure are not limited thereto, and elements can be modified within a range not departing from the gist of the disclosure, when the disclosure is practiced. Further, constituent elements disclosed in the above embodiments can be suitably combined. For example, some of the constituent elements of the above-described embodiments may be omitted described. Further, different embodiments and modifications may be combined as appropriate. Any one of the above-described operations may be performed in various other ways, for example, in an order different from the one described above.

1. An electromechanical transducer element comprising:
a base substrate;
a first electrode on the base substrate;
a piezoelectric body on the first electrode;
a second electrode on the piezoelectric body; and
the base substrate having a void area opposite to the piezoelectric body via the first electrode,
the void area having a width on a cross section cut along a layer direction of the electromechanical transducer element, the width satisfying
0.65≤Pw/Cw≤0.95
where Cw represents the width of the void area, and Pw represents a width of the piezoelectric body on the cross section.

2. The electromechanical transducer element according to claim 1,
wherein, on the cross section cut along the layer direction, the second electrode has a width that satisfies
0.6≤Uw/Pw≤0.9
where Uw represents the width of the second electrode on the cross section, and Pw represents the width of the piezoelectric body on the cross section.

3. The electromechanical transducer element according to claim 1,
wherein the piezoelectric body includes a center portion thicker than a peripheral portion of the piezoelectric body in the layer direction.

4. The electromechanical transducer element according to claim 3,
wherein the second electrode satisfies
0.2≤Pte/Ptc≤1.0
where Pte represents a thickness of the piezoelectric body at an end of the second electrode, and Ptc represents a thickness of the center portion of the piezoelectric body.

5. The electromechanical transducer element according to claim 3,
wherein the center portion of the piezoelectric body has a thickness satisfying
1 μm≤Ptc≤4 μm
where Ptc represents the thickness of the center portion of the piezoelectric body.

6. The electromechanical transducer element according to claim 1,
wherein respective shapes of the piezoelectric body and the void area viewed along the layer direction are axisymmetric with respect to a center axis parallel to the layer direction, the center axis passing through a center of the piezoelectric body.

7. The electromechanical transducer element according to claim 1, comprising:
a plurality of piezoelectric bodies disposed in an array; and
a plurality of void areas disposed in an array.

8. An ultrasonic transducer comprising the electromechanical transducer element according to claim 1.

9. An ultrasonic probe comprising:
the ultrasonic transducer according to claim 8;
an acoustic lens; and
a support supporting the ultrasonic transducer and the acoustic lens,
wherein the ultrasonic probe is configured to:
apply voltage to the electromechanical transducer element to vibrate an end wall of the void area opposite an open side of the void area, to generate an ultrasonic wave, and
detect a vibration of the ultrasonic wave reflected from a measurement target.

10. An ultrasonic diagnostic apparatus comprising:
the ultrasonic probe according to claim 9; and
a display configured to display a shape of a measurement target.

11. A method for manufacturing an electromechanical transducer element, the method comprising:
forming a first electrode on a base substrate;
forming, by an inkjet method, a piezoelectric body on the first electrode;
forming a second electrode on a portion of the piezoelectric body; and
forming a void area in the base substrate on a side opposite the piezoelectric body via the first electrode,
the void area having a width on a cross section cut along a layer direction of the electromechanical transducer element, the width satisfying
0.65≤Pw/Cw≤0.95
where Cw represents the width of the void area, and Pw represents a width of the piezoelectric body.
